(19) United States
(12) Patent Application Publication
(10) Pub. No.: US 2022/0087257 A1
(43) Pub. Date: Mar. 24, 2022

(54) Anti-Microbial Agent

(71) Applicants: Sidharth Sareen, La Mirada, CA (US); Mukul Sareen, Ludhiana (IN); Jasbeer Sareen, Ludhiana (IN)

(72) Inventors: Sidharth Sareen, La Mirada, CA (US); Mukul Sareen, Ludhiana (IN); Jasbeer Sareen, Ludhiana (IN)

(21) Appl. No.: 17/481,651

(22) Filed: Sep. 22, 2021

Related U.S. Application Data
(60) Provisional application No. 63/082,374, filed on Sep. 23, 2020.
(60) Provisional application No. 63/082,849, filed on Sep. 24, 2020.

(51) Int. Cl. A01N 37/02; A61K 8/36; A61Q 17/00; A01P 1/00; A01N 25/12; C09D 5/14; C09D 175/06

(52) U.S. Cl. CPC A01N 37/02; A61K 8/361; A61Q 17/005; C09D 175/06; A01N 25/12; C09D 5/14; A01P 1/00

(57) ABSTRACT

An Anti-Microbial Agent (AMA) kills viruses, bacteria, and fungi on the surface and prevents cross-transmission of the diseases by providing products using a natural AMA having a fast acting, long-term virucidal property. The AMA includes fatty acids from natural plants is an extract which may be added to plastic objects like laptops, handles, mobile phones and covers, suitcases, hand rails, plastic bags, medical apparels, plastic gloves or products that are handled multiple times. The AMA may be converted to coatings which may be applied by spray, roller, dip or any other way to inanimate objects such as fabrics, paper and surfaces to make them anti-microbial. The AMA may be combined with cosmetics and applied on the body, for example on hands, to avoid pathogens from living on hands and causing cross-contamination. The AMA can also be applied during the washing of garments.

### CROSS-REFERENCE TO RELATED APPLICATIONS

The present application claims the priority of U.S. Provisional Patent Application Ser. No. 63/082,374 filed Sep. 23, 2020, and U.S. Provisional Patent Application Ser. No. 63/082,849 filed Sep. 24, 2020, which applications are incorporated in its entirety herein by reference.

### BACKGROUND OF THE INVENTION

The present invention relates anti-microbial agents and in particular to anti-microbial agents which may be incorporated into plastics, coated on a variety of substrates, incorporated into natural and synthetic materials or paints, and used with cosmetics, detergents or washing additives for garments.

The surfaces of objects and clothing can carry a variety of pathogens and can cause cross-transmission of diseases. An infected person may touch various surfaces leaving behind pathogens which can stay alive on surfaces for many hours to days. Currently available technologies do not provide anti-microbial products which are fast acting and also natural. Such currently available technology utilizes silver ions or nano-particles, copper iodide, copper ions or synthetic anti-microbials etc. based on pesticides and biocides which are much slower acting and may be considered harmful for human contact, inhalation and ingestion.

### BRIEF SUMMARY OF THE INVENTION

The present invention addresses the need for a safe, natural & fast acting Anti-Microbial Agent (AMA) according to the present invention which is able to kill viruses, bacteria and fungi on surfaces and prevent cross-transmission of diseases by providing a range of products using a natural AMA having a fast acting, long-term virucidal property. The AMA is a fatty acids obtained from natural plants, seeds, nuts or flowers and may be applied to laptops, handles, mobile phones, mobile phones covers, suitcases, hand rails and products that are handled multiple times. The AMA may be converted to a coating which may be applied by spray, roller, dip or any other way to inanimate objects such as fabrics, paper and surfaces to make them anti-microbial. The AMA may further be combined with cosmetics and applied to the body, for example on hands, to avoid pathogens from living on hands and causing cross-contamination. The AMA may also be formulated into detergents or additives to be applied on garments during washing process.

In accordance with one aspect of the invention, there is provided a natural plant based AMA having low toxicity for humans and capable to kill/reduce viruses by over 99% within the first minute of contact.

In accordance with another aspect of the invention, there is provided an AMA which may be added to various polymers directly, or first formulated into an additive masterbatch including the AMA as a functional additive. The AMA functional additive, a combination of functional additives including the AMA, is then added while processing plastic products to make various plastic objects like disposable gloves, suitcases, toilet seats, door handles, courier/mailer bags etc., computers, keyboards, phones etc.

In accordance with yet another aspect of the invention, there is provided an AMA coating formed using the natural ingredients which may be mixed with special polymers or coated onto fabrics used to make medical provider coveralls, travel jackets, airline seat covers, etc.

In accordance with still another aspect of the invention, there is provided an AMA which can be combined with cosmetics providing anti-microbial protection to the applied areas for a significant amount of time. The cosmetics found to have no adverse effect, rash, or irritation.

In accordance with another aspect of the invention, there is provided a natural AMA which is harmless if digested in small dosages or on hands, for example, in case of cosmetics, safe for human contact, and safe for the environment.

In accordance with another aspect of the invention, there is provided a natural AMA which can be added to paint like finishes and applied to surfaces to provide an anti-microbial property to walls, doors, door handles, floor, etc.

In accordance with still another aspect of the invention, the AMA is preferably incorporated between 0.1% and 35% in additive masterbatches and more preferably between 0.1% and 10% in additive masterbatches, and preferably between 0.1% and 15% directly into plastic objects, and preferably between 0.1% and 20% in surface coatings or paints, and preferably between 0.1% and 100% when directly applied onto surfaces, fabrics, inanimate objects or as wash treatments.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

Where the terms “about” or “generally” are associated with an element of the invention, it is intended to describe a feature's appearance to the human eye or human perception, and not a precise measurement, or typically within 10 percent of a stated value. Percentages of ingredients are by weight.

The present invention is an Anti-Microbial Agent (AMA) suitable for preparing compositions which destroy viruses on contact. The AMA comprises naturally obtained fatty acids having anti-microbial properties such as octanoic acid, nonanoic acid, decanoic acid, undecylic acid dodecanoic acid, hexanoic acid, and heptanoic acid, and is preferably octanoic acid. AMA compositions are prepared as follows.

### Additive Masterbatches including the AMA

An additive masterbatch including an Anti-Microbial Agent (AMA). A polymer additive masterbatch is prepared using an extruder. The AMA, for example octanoic acid, makes up preferably between 0.1% and 35% and more preferably between 0.1% and 10% of the additive masterbatch to provide a stable masterbatch, of polymer granules and other inert ingredients in a mixer and is extruded to form a concentrate additive masterbatch containing the AMA. The additive masterbatch can be transported to various customers who would use this as a concentrate to make various products. The additive masterbatch may be added at dosage levels ranging from 0.5% to 30% to polymer products depending upon the wall thickness and the manufacturing process, for example, extrusion, injection molding, rotational molding or blow molding or any other known process. The additive masterbatch may include different polymers like PE, PP, ABS, PC, Nylon, TPU, EVA, PVC, PBT, etc. The combination of the additive masterbatch at recommended dosage levels with other polymers, may then be formed into products like gloves, furniture, mailer bags, medical products, disposable packaging, travel products like suitcases, door handles and objects that can be a source of cross-transmission of the virus or pathogens.

### Coatings Including the AMA

A coating is formulated using the AMA dissolved in a solvent such as acetone. The coating may be coated onto non-woven fabrics, paper as well as cotton and polyester fabrics to make anti-microbial fabrics. To improve the binding and have a better carrier of the AMA, a thermoplastic urethane may be added into the solvent with the AMA and then the coating formed may be applied onto the surface of the fabrics. Another coating may be formulated using toluene as solvent and silicone as an inert carrier/binder on the surface of the fabric. The solvent and the binder can be changed keeping the same AMA depending upon the application, fabric and end use requirement. The AMA is preferably between 2.5% and 15% of the coating, and more preferrably between 5% and 10% of the coating, and is most preferably 10% of the coating.

### Cosmetics Including the AMA

1) A first hand sanitizer containing more than 70% alcohol is formulated with between 0.1% and 3% AMA.

2) A second hand washing liquid is formulated using between 0.1% and 5% of the AMA.

3) A moisturizer is formulated using between 0.3% and 7.5% of the AMA.

4) A skin cream is prepared by addition of between 0.1% and 7.5% of the AMA.

### Paints Including the AMA

An AMA paint is prepared using Polyurethane as the medium and preferably about 10% AMA as the anti-microbial agent. The AMA paint may be applied onto steel objects like door handles as well as a wooden surface and checked for anti-viral activity. The AMA paint was found to be effective at greater than 99% within the first minute. The AMA paint may further be a clear coat.

### Washing Agents Including the AMA

A garment wash and washing additive is manufactured preferably including between 0.1% and 15% AMA and more preferably between 4% and 5% AMA. The garment wash leaves the coating on the garments after washing which was effective in virucidal function.

### Polymeric Articles Including the AMA

A polymeric article is manufactured by direct mixing of the AMA at preferably between 0.1% and 4%, and more preferably about 0.5%, with the Polypropylene granules.

### EXAMPLE 1

In one embodiment of the present invention, an Anti-Microbial polymer masterbatch is provided. This can be used to make various products by mixing with base polymers to products ranging from plastic films or sheets, gloves, bags, sheets, non-woven coveralls, solid objects like laptop bodies, covers for keyboards, covers for cell phones, airline seat covers etc. The AMA can be mixed with polymers, waxes and other ingredients commonly used to compound a masterbatch. The concentration of the AMA can be between 0.1% and 25% and more preferably between 0.1% and 10% to provide a stable masterbatch. The masterbatch was added to a plastic film or sheet during its extrusion process at dosage level of 10% of the outer layer. Remaining 90% was a 1:1 mixture of Linear Low Density Polyethylene obtained from Exxon Mobile grade 1018 and High Density Polyethylene and converted to a 50 micron polymeric film or sheet. This film or sheet obtained was for the purpose of general shopping bags and when tested for virucidal activity, it was able to eradicate 99.16% of viruses within 1 minute.

### EXAMPLE 2

In another embodiment of the present invention, a polymeric article is manufactured by direct mixing of the AMA at 0.3% with the Polypropylene granules and then injection molded into a lunch box. The box is checked for virucidal activity and is found to destroy greater than 99% of the viruses within 1 minute.

### EXAMPLE 3

In another embodiment a plastic film or sheet for gloves application having 20 micron thickness and 15% AMA additive masterbatch (as in example 1) was made by directly mixing the AMA with the polymer and was tested for virucidal activity. This film or sheet was for the use of making plastic disposable gloves. It was found that the formed film or sheet was able to reduce viruses by more than 99% within 1 minute.

### EXAMPLE 4

In another embodiment a plastic film or sheet for shopping bag application having 50 micron thickness and 0.5% AMA was made by directly mixing the AMA with the polymer and was tested for virucidal activity. It was found that the formed film or sheet was able to reduce viruses by more than 99% within 1 minute.

### EXAMPLE 5

In another embodiment the AMA was mixed directly at 0.5% with the polymer and converted to a plastic cling film or sheet for suit cases and other inanimate objects or food wrapping application. This film or sheet may also contain cling agents. This film or sheet was tested to have virucidal effect by reducing 99.2% viruses within the first 1 minute.

### EXAMPLE 6

In yet another embodiment of the present invention, a polymer coating was manufactured using thermoplastic urethane polymer and the AMA at 5% concentration and coated on various fabrics such as polypropylene non-woven sheet, face shield, N95 Mask, polyester fabric and cotton fabric. These fabrics and products were then sent for testing for virucidal activity and was found that the coating was able to neutralize the virus by more than 99% for all surfaces. It was also found that the efficacy on the fabric was maintained even after 15 washes. The virus selected in all tests was MS2 which is safe to handle in the laboratory and is considered to have a stronger structure compared to Covid 19 virus. The general understanding in the medical world is that if an anti-microbial substance can eradicate MS2 virus it will work similar or better for Covid19 virus. The virucidal coating can be made using other polymers or also directly coating the active anti-microbial agent onto garments or fabrics or inanimate objects.

### EXAMPLE 7

In another embodiment a coating was manufactured using polyurethane based coating and addition of the AMA at between 2.5% and 15% concentration. This coating can be used on floors, walls, metal surfaces, wooden surfaces to work as anti-microbial and provide virucidal action. The coating was also tested for virucidal activity and found to reduce the virus by more than 99% in 1 minute in both the cases.

### EXAMPLE 8

In yet another embodiment of the present invention a skin cream, an alcohol based skin sanitizer, a surface disinfectant, a detergent skin wash and a skin lotion and moisturizer were prepared using the AMA. All the products made were tested in house and found to be effective & mild on hands during use. The AMA was tested at between 0.1% and 7.5% in each of the products and could be increased depending upon the application.

### EXAMPLE 9

In yet another embodiment an anti-corrosion anti-viral plastic film is prepared by adding a masterbatch containing AMA and vapor corrosion inhibitors such as dicyclohexylamine nitrite, sodium benzoate, cyclohexylamine benzoate, and dicyclohexylammonium benzoate. The vapor corrosion inhibitors are substances that prevent corrosion of metal and are added as chemicals to plastic sheets. When a metallic component is packed in such a plastic sheet it remains protected from corrosion. A plastic sheet combines properties of corrosion inhibition along with Anti Viral features. The masterbatch is then mixed at 10% with 90% Polyethylene granules and extruded to a plastic film or sheet. The film or sheet was tested for Corrosion Resistance as per Vapor Inhibiting Ability Test TL 8135 as well as German Razor blade test and was found to pass both the tests along with ISO 21702 which measures anti-Viral Activity on Plastics and was able to reduce viruses by more than 95% in the first 30 seconds & more than 99.16% within the first minute.

### EXAMPLE 10

In yet another embodiment a carton box is made by first coating the paper with thermoplastic polyurethane dissolved in acetone and in the AMA and then converted to a corrugated box. This box exhibited Anti-Viral properties on the surface by eradicating more than 99% within the first 1 minute.

While the invention herein disclosed has been described by means of specific embodiments and applications thereof, numerous modifications and variations could be made thereto by those skilled in the art without departing from the scope of the invention set forth in the claims.

I claim:

1. An Anti-Microbial Agent (AMA) combination comprising:
a plant based naturally obtained anti-microbial; and
a combination selected from the group consisting of:
the AMA combined with polymer granules to produce an additive masterbatch;
the AMA directly combined with polypropylene to manufacture polymeric articles.
the AMA dissolved in a solvent to make a coating for treatment of surfaces and fabrics;
the AMA combined with a hand sanitizer, a hand soap, a moisturizer, or a skin cream to provide a cosmetic;
the AMA is combined with polyurethane to provide a paint; and
a washing agent including the AMA.

2. The AMA combination of claim 1, wherein the anti-microbial is a fatty acid having anti-microbial properties.

3. The AMA combination of claim 2, wherein the fatty acid is naturally obtained.

4. The AMA combination of claim 3, wherein the fatty acids are obtained from natural plants, seeds, nuts or flowers.

5. The AMA combination of claim 2, wherein the fatty acid is selected from the group consisting of octanoic acid, nonanoic acid, decanoic acid, undecylic acid, dodecanoic acid, hexanoic acid, and heptanoic acid.

6. The AMA combination of claim 5, wherein the fatty acid is the octanoic acid.

7. The AMA combination of claim 1, wherein the AMA is between 0.1% to 30% of the additive masterbatch for polymer products.

8. The AMA combination of claim 7, wherein the additive masterbatch is added at dosage levels between 0.1% to 10% for the polymer products.

9. The AMA combination of claim 1, wherein the polymeric articles are 0.3% AMA mixed with polypropylene granules.

10. The AMA combination of claim 1, wherein the AMA dissolved in the solvent to make the coating.

11. The AMA combination of claim 10, wherein the AMA is about 10% of the coating.

12. The AMA combination of claim 1, wherein the AMA combined with a hand sanitizer, a hand soap, a moisturizer, or a skin cream to provide the cosmetic.

13. The AMA combination of claim 12, wherein the AMA is between 0.1% and 7.5% of the cosmetic.

14. The AMA combination of claim 1, wherein the AMA combined with a medium to provide a paint.

15. The AMA combination of claim 14, wherein the AMA is about 10% of the paint and the medium is polyurethane.

16. The AMA combination of claim 1, wherein the AMA combined with a washing additive leaving a virucidal coating on garments.

16. The AMA combination of claim 15, wherein the AMA is between 4% and 5%.

17. An Anti-Microbial Agent (AMA) combination comprising:
a plant based naturally obtained anti-microbial selected from the group consisting of octanoic acid, nonanoic acid, decanoic acid, undecylic acid, dodecanoic acid, hexanoic acid, and heptanoic acid;
a combination selected from the group consisting of:
the AMA combined with polymer granules to produce an additive masterbatch;
the AMA directly combined with polypropylene to manufacture polymeric articles.
the AMA dissolved in a solvent to make a coating for treatment of surfaces and fabrics;
the AMA combined with a hand sanitizer, a hand soap, a moisturizer, or a skin cream to provide a cosmetic;
the AMA is combined with polyurethane to provide a paint; and
a washing agent including the AMA.

18. An Anti-Microbial Agent (AMA) combination comprising:
octanoic acid;
a combination selected from the group consisting of:
between 0.1% to 30% of the AMA is combined with polymer granules to produce an additive masterbatch;
between 0.1% to 10% of the AMA is combined with polypropylene to manufacture polymeric articles.
between 2.5% and 15% of the AMA dissolved in a solvent to make a coating for treatment of surfaces and fabrics;
between 0.1% and 7.5% of the AMA is combined with a hand sanitizer, a hand soap, a moisturizer, or a skin cream to provide a cosmetic;
10% of the AMA is combined with polyurethane to provide a paint; and
a washing agent including 0.3% and 0.7% of the AMA.
